(19) United States
(12) Patent Application Publication
(10) Pub. No.: US 2025/0249108 A1
(43) Pub. Date: Aug. 7, 2025

(54) PRODRUGS FOR CANCER TREATMENT

(71) Applicant: GATC Health Corp, Irvine, CA (US)

(72) Inventors: Ian Jenkins, Irvine, CA (US); Eric J. Mathur, Encinitas, CA (US); Jonathan Lakey, Irvine, CA (US); Robert Tinder, Tacoma, WA (US); Vaishnavi Narayan, Pittsburgh, PA (US); Jayson Uffens, Draper, UT (US)

(73) Assignee: GATC Health Corp, Irvine, CA (US)

(21) Appl. No.: 18/855,286

(22) PCT Filed: Apr. 8, 2023
(86) PCT No.: PCT/US2023/017988

§ 371 Date: Oct. 8, 2024

Related U.S. Application Data
(60) Provisional application No. 63/329,272, filed on Apr. 8, 2022.

(51) Int. Cl. A61K 47/55

(52) U.S. Cl. CPC A61K 47/551

(57) ABSTRACT

Embodiments include prodrug compounds, that can be used, inter alia, for treating an ailment such as cancer. The prodrug compounds can include an active agent component such as a cyclic monoterpene. The compounds can also include a prodrug component such as cholecalciferol or retinoic acid. Benefits of the prodrug compounds include increased solubility, increased half-lives, increased stability, increased lipophilicity, improved targeting (e.g., to tumor cells) and/or increased permeability/transmission across the blood-brain barrier.

### RELATED APPLICATIONS

This application claims priority to U.S. provisional patent application Ser. No. 63/329,272 filed on Apr. 8, 2022. The contents of the aforementioned application are incorporated herein by reference.

### FIELD OF THE INVENTION

The invention relates generally to small molecule therapeutics, and more specifically, to prodrug compounds with high bioavailability and antineoplastic activity.

### BACKGROUND

Cancer can be defined as a group of diseases that involve abnormal cell growth with the potential to invade or spread to other parts of the body. Despite advances in technology, cancer continues to be a significant cause of death and incalculable suffering. Cancer has been linked to several factors including smoking, obesity, poor diet, lack of physical activity and excessive consumption of alcohol. Other factors include certain infections, exposure to ionizing radiation and environmental pollutants. Certain cancers have been linked to infections such as Helicobacter pylori, hepatitis B, hepatitis C, human papillomavirus infection, Epstein-Barr virus and human immunodeficiency virus (HIV). Cancer is the second most common cause of death in the United States.

Patients with cancer often have limited treatment options. Conventional cancer treatments are directed at removing cancerous tissue and preventing it from spreading. Despite advances in research, these treatments have remained relatively unchanged in recent decades. Treatment can include a combination of surgery, radiation therapy, chemotherapy, targeted therapy, hormonal therapy and palliative care. Treatments are usually pursued based on the type, location and grade of the cancer as well as the patient's health and preferences. However, these options have limitations. They can be ineffective, particularly when cancer has metastasized.

Because cancer cells divide faster than most normal cells, they can be sensitive to chemotherapy drugs. However, chemotherapy drugs will also attack other cells in the body, especially fast-dividing cells such as blood cells and the cells lining the mouth, stomach, and intestines. Radiation and chemotherapy typically lead to multiple side-effects, especially epithelial cell damage. Accordingly, there is a need for improved medications and methods of treating cancer that are more targeted and have less deleterious side effects.

Recent efforts have been directed toward small molecule medicaments. Perillyl alcohol (PA) is a monoterpene that has been used orally for the treatment of systemic cancer. Perillyl alcohol is a metabolite of limonene, which itself is formed from geranyl pyrophosphate in the mevalonate pathway. Conversion of limonene to perillyl alcohol is accomplished via hydroxylation by enzymes that belong to the superfamily of cytochrome P450 proteins. Perillyl alcohol can be metabolized further to perillaldehyde (perillyl aldehyde) and perillic acid.

Published in vitro studies have demonstrated that perillyl alcohol (PA) and limonene can both induce apoptosis in cancer cells. Moreover, under specific conditions, these compounds can restore cancer cell identity without adversely affecting the healthy cells within the cell culture population. However, PA and limonene have not been used in clinical settings and patient care due to their poor bioavailability and issues with ADME (adsorption, distribution, metabolism and excretion). When taken orally, the compounds negatively impact mucosal tissues and generally have short half-lives. Common side effects include nausea, diarrhea and abdominal pain.

Due to the shortcomings of the use of PA as described above, there is a need for improved formulations and methods of administering the compound. Such formulations and methods should improve its bioavailability and ADME. Aspects of the present invention fulfill these needs and provide further related advantages as described in the following summary.

### SUMMARY OF THE INVENTION

Aspects of the present disclosure teach certain benefits in construction and use which give rise to the exemplary advantages described below.

The present disclosure solves the problems described above by providing prodrugs of perillyl alcohol (PA), limonene, perillic acid, perillyl alcohol acetate, perillic acid methyl ester, perillyl alcohol isobutyrate, perillic acid ethyl ester, perillyl alcohol pivaloate, perillic acid isopropyl ester, perillyl perilliate, perillyl carbonate and/or other monocyclic terpenes. In embodiments, the prodrugs can be used to treat ailments such as cancer. In embodiments, the prodrugs can cross the blood-brain barrier. In embodiments, the prodrugs have one or more of increased solubility, increased half-lives, increased stability, increased lipophilicity, improved targeting (e.g., to tumor cells), increased protection from metabolic inactivation/elimination, improved patient compliance and increased permeability/transmission across the blood-brain barrier.

The prodrugs can increase bioavailability and improve ADME (adsorption, distribution, metabolism and excretion) of medicaments/agents such as monocyclic terpenes.

The disclosure also relates methods of chemical synthesis of prodrugs. In embodiments, the prodrug includes cholecalciferol/Vitamin D, retinoic acid, N-methyl perrilic acid, tyrosine and/or valproic acid.

In embodiments the prodrugs protect the mucosae from effects of medicaments/agents. In embodiments, the prodrugs enable targeted delivery to tumor cells, where the medicaments/agents induce apoptosis of the tumor cells. In embodiments, the prodrugs can cross the blood-brain barrier.

Accordingly, embodiments include small molecule therapeutics that include an active agent (e.g., a monocyclic terpene) and a prodrug component. A therapeutic amount of the composition can be administered to treat an ailment such as cancer.

Embodiments include a therapeutic that includes perillic acid and cholecalciferol. A therapeutic amount of the composition can be administered to treat an ailment such as cancer.

Embodiments include a therapeutic that includes limonene and cholecalciferol. A therapeutic amount of the therapeutic can be administered to treat an ailment such as cancer.

Embodiments include a therapeutic that includes limonene, PA, perillic acid and cholecalciferol. A therapeutic amount of the composition can be administered to treat an ailment such as cancer.

Embodiments include a therapeutic that includes PA, limonene and cholecalciferol. A therapeutic amount of the composition can be administered to treat an ailment such as cancer.

Embodiments include a therapeutic that includes PA, retinoic acid and cholecalciferol. A therapeutic amount of the composition can be administered to treat an ailment such as cancer.

Embodiments include a therapeutic that includes PA and retinoic acid. A therapeutic amount of the composition can be administered to treat an ailment such as cancer.

Embodiments include a therapeutic that includes perillic acid and retinoic acid. A therapeutic amount of the composition can be administered to treat an ailment such as cancer.

Embodiments include a therapeutic that includes limonene and retinoic acid. A therapeutic amount of the composition can be administered to treat an ailment such as cancer.

Embodiments include a therapeutic that includes limonene, PA, perillic acid and retinoic acid. A therapeutic amount of the composition can be administered to treat an ailment such as cancer.

Embodiments include a therapeutic that includes PA, limonene and retinoic acid. A therapeutic amount of the composition can be administered to treat an ailment such as cancer.

Embodiments include a therapeutic that includes one or more of perillyl alcohol, perillic acid, perillyl alcohol acetate, perillic acid methyl ester, perillyl alcohol isobutyrate, perillic acid ethyl ester, perillyl alcohol pivaloate, perillic acid isopropyl ester, perillyl perilliate and perillyl carbonate. The compound can enter a target cell via passive transport.

Embodiments include a prodrug that uses active transport to enter a target cell. The prodrug can include N-methyl perillic acid, tyrosine, valproic acid, retinoic acid or Vitamin D.

In embodiments, the compounds described herein are administered to treat one or more of leukemia, lung cancer, breast cancer, colon cancer, colorectal cancer, prostate cancer, renal cell carcinoma.

Embodiments also include methods of treating cancer, such as glioblastoma with chemotherapy, where the cells are treated with an effective amount of a monoterpene derivative and then exposed to chemotherapy. Monoterpene derivative treatment can be before, during and/or after chemotherapy.

In embodiments, the compounds described herein are administered to treat ailments such as infection, gallstone, cholecystitis, gastroesophageal reflux (GERD) and angiocholitis.

In embodiments, the compounds described herein are administered to restore cell identity in cells of glioblastoma and/or astrocytoma.

In embodiments, the compounds described herein are administered to increase apoptosis in glioblastoma and/or astrocytoma cells.

In embodiments, the compounds described herein are administered to maintain homeostasis of healthy neuronal cells and astrocytes.

In embodiments, the compounds described herein are administered to treat Parkinson's disease by removing amyloid plaques in brain tissue.

Embodiments also include methods of treating an ailment. The method can include combining a medicament/agent (e.g., limonene, PA and/or perillic acid) with another molecule (e.g., cholecalciferol or retinoic acid) to create a prodrug that has greater bioavailability and/or improved ADME (adsorption, distribution, metabolism and excretion). The prodrug can be administered to treat an ailment such as cancer.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A shows chemical structures of isomers of perillyl alcohol (PA).

FIG. 1B shows the stepwise oxidation of limonene to perillic acid.

FIG. 2 shows the chemical structures of cholecalciferol and retinoic acid.

FIG. 3 shows the stepwise biosynthesis of cholecalciferol (vitamin D3).

FIG. 4 shows chemical structures of active transport prodrugs according to some embodiments.

FIG. 5 shows chemical structures of passive transport prodrugs according to some embodiments.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “neoplasia” refers to a disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “glioma” refers to a type of tumor that starts in the glial cells of the brain or the spine. Gliomas comprise about 30 percent of all brain tumors and central nervous system tumors, and 80 percent of all malignant brain tumors.

The term “glioblastoma” refers to an aggressive type of cancer that can occur in the brain or spinal cord. Glioblastoma forms from cells called astrocytes that support nerve cells. Glioblastoma can occur at any age but tends to occur more often in older adults. Initially, signs and symptoms of glioblastoma are nonspecific. They may include headaches, personality changes, nausea, and symptoms similar to those of a stroke. Symptoms often worsen rapidly and may progress to unconsciousness. The cause of most cases of glioblastoma is not known. Uncommon risk factors include genetic disorders, such as neurofibromatosis and Li-Fraumeni syndrome, and previous radiation therapy. Glioblastomas represent 15% of all brain tumors. They can either start from normal brain cells or develop from an existing low-grade astrocytoma. The diagnosis typically is made by a combination of a CT scan, MRI scan, and tissue biopsy. Treatment usually involves surgery, after which chemotherapy and radiation therapy are used. The medication temozolomide is frequently used as part of chemotherapy. High-dose steroids may be used to help reduce swelling and decrease symptoms. Surgical removal of the tumor is linked to increased survival but only for a duration of months.

The term “astrocytoma” refers to a type of brain tumor. They originate in a particular kind of glial cells, star-shaped brain cells in the cerebrum called astrocytes. This type of tumor does not usually spread outside the brain and spinal cord and it does not usually affect other organs. Astrocytomas are the most common glioma and can occur in most parts of the brain and occasionally in the spinal cord. Within the astrocytomas, two broad classes are generally recognized, those with (a) narrow zones of infiltration (mostly noninvasive tumors; e.g., pilocytic astrocytoma, subependymal giant cell astrocytoma, pleomorphic xanthoastrocytoma), that often are clearly outlined on diagnostic images and (b) diffuse zones of infiltration (e.g., high-grade astrocytoma, anaplastic astrocytoma, glioblastoma), that share various features, including the ability to arise at any location in the central nervous system, but with a preference for the cerebral hemispheres; they occur usually in adults, and have an intrinsic tendency to progress to more advanced grades. People can develop astrocytomas at any age. The low-grade type is more often found in children or young adults, while the high-grade type is more prevalent in adults. Astrocytomas in the base of the brain are more common in young people and account for roughly 75% of neuroepithelial tumors.

The term “cell identity” can be defined by the expression, or activity, of certain genes in its DNA and the resulting production of particular proteins. Increased biological complexity is enabled by the ability of cells to differentiate and attain distinct “identities” within a system—reflecting a divergence in form or function from precursor cells. This identity has largely been defined in terms of cell type and cell state. Precise definitions for these terms remain elusive: cell type has historically been described by observing reproducible functional distinctions in vivo or in vitro (often coupled with expression of a set of marker genes), while cell state refers to dynamic, responsive changes that alter the phenotype and function of the cell, but not so significantly that a new cell type is acquired.

The term “blood-brain barrier” or “BBB” refers to a highly selective semipermeable border of endothelial cells that prevents solutes in the circulating blood from non-selectively crossing into the extracellular fluid of the central nervous system where neurons reside. The blood-brain barrier is formed by endothelial cells of the capillary wall, astrocyte end-feet ensheathing the capillary, and pericytes embedded in the capillary basement membrane. This system allows the passage of some small molecules by passive diffusion, as well as the selective and active transport of various nutrients, ions, organic anions, and macromolecules such as glucose and amino acids that are crucial to neural function

The term “perillyl alcohol,” “PA” or “POH” refers to a naturally occurring monoterpene. It is also identified (i.e., IUPAS name) as [4-(prop-1-en-2-yl)cyclohex-1-en-1-yl]methanol). PA and its precursor limonene are naturally occurring monocyclic terpenes that can be derived from the mevalonate pathway in plants. PA has also shown some antitumor activity in laboratory and animal studies. Clinical trials in humans have been inconclusive with adverse side effects. PA can be found in the essential oils of various plants, such as lavender, lemongrass, sage, and peppermint. It has many manufacturing, household and medical applications.

The term “perillic acid” refers to a metabolite/conjugate acid of perillyl alcohol. Perillic acid is an alpha, beta-unsaturated monocarboxylic acid and a cyclohexenecarboxylic acid. It has a role as an antineoplastic agent, a human metabolite and a mouse metabolite.

The term “limonene” refers to colorless liquid aliphatic hydrocarbon classified as a cyclic monoterpene. It is the major component in the oil of citrus fruit peels. It is also identified (i.e., IUPAS name) as 1-Methyl-4-(prop-1-en-2-yl)cyclohex-1-ene.

The term “cholecalciferol” or “vitamin D3” refers to an analog of vitamin D. It is naturally synthesized in skin and functions as a pro-hormone, being converted to calcitriol. It is converted in the liver to calcifediol (25-hydroxyvitamin D) which is then converted in the kidney to calcitriol (1,25-dihydroxyvitamin D). One of its actions is to increase the uptake of calcium by the intestines.

The term “prodrug” refers to a derivative of a known direct acting drug, which derivative has enhanced delivery characteristics and therapeutic value as compared to the drug and is transformed into the active drug by an enzymatic or chemical process.

The term “terpene” refers to a class of compounds with the formula (C₅H₈)ₙ. Terpenes include more than 30,000 compounds that are produced predominantly by plants, particularly conifers. Terpenes can be further classified by the number of carbons (e.g., monoterpenes (C₁₀), sesquiterpenes (C₁₅), diterpenes (C₂₀), etc.). Terpenes include a diverse group of naturally occurring compounds. They are mostly found in plants and form the major constituent of essential oils from plants. Terpenes have a wide range of medicinal uses among which anti-plasmodial activity is notable as its mechanism of action is similar to the popular antimalarial drug in use (i.e., chloroquine). Monoterpenes specifically are widely studied for their antiviral property. With growing incidents of cancer and diabetes in modern world, terpenes also have the potential to serve as anticancer and antidiabetic reagents.

The term “monoterpene” refers to a class of terpenes that includes two isoprene units and have the molecular formula C₁₀H₁₆ such as perillyl alcohol. Monoterpenes can be linear (i.e., acyclic) or contain rings (i.e., cyclic). Monoterpenes can be modified by oxidation or rearrangement to produce monoterpenoids. Examples of monoterpenes and monoterpenoids include geranyl pyrophosphate, ocimene, myrcene, geraniol, citral, citronellal, linalool, limonene, terpinenes, phellandrenes and terpinolene.

The term “retinoic acid” (used simplified here for all-trans-retinoic acid) is a metabolite of vitamin A1 (all-trans-retinol) that mediates the functions of vitamin A1 required for growth and development. All-trans-retinoic acid is required in chordate animals, which includes all higher animals from fish to humans. All-trans-retinoic acid (ATRA) is the major occurring retinoic acid, while isomers like 13-cis- and 9-cis-retinoic acid are also present in much lower levels in mammals.

The term “mevalonate pathway” refers to a metabolic pathway which plays a key role in multiple cellular processes by synthesizing sterol isoprenoids, such as cholesterol, and non-sterol isoprenoids, such as dolichol, heme-A, isopentenyl tRNA and ubiquinone.

The term “linker” refers to a functional group that covalently bonds two or more moieties in a compound or material. For example, the linker can serve to covalently bond a prodrug moiety to an active agent.

The term “alkyl” refers to a straight or branched, saturated, aliphatic radical having the number of carbon atoms indicated, Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, Cia alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy. The term “alkylene” refers to a divalent alkyl radical.

The term “heteroalkyl” refers to an alkyl group as described herein, wherein one or more carbon atoms are optionally and independently replaced with heteroatom selected from N, O, and S. The term “heteroalkylene” refers to a divalent heteroalkyl radical.

The term “amino” refers to a moiety —NR₃, wherein each R group is H or alkyl. An amino moiety can be ionized to form the corresponding ammonium cation.

The term “hydroxy” refers to the moiety —OH.

The term “carboxy” refers to the moiety —C(O)OH. A carboxy moiety can be ionized to form the corresponding carboxylate anion.

The term “amido” refers to a moiety —NRC(O)R or —C(O)NR₂, wherein each R group is H or alkyl.

The term “nitro” refers to the moiety —NO₂.

The term “oxo” refers to an oxygen atom that is double-bonded to a compound (i.e. O═).

The term “formulation” as used herein refers to the prodrug compositions disclosed herein and excipients combined together which can be administered for therapeutic use. The formulation can optionally include other agents such as, for example, water, emulsions, buffering agents and preservatives.

The term “passive transport” refers to a type of membrane transport that does not require energy to move substances across cell membranes. The four main kinds of passive transport are simple diffusion, facilitated diffusion, filtration, and/or osmosis. Passive transport follows Fick's first law and the second law of thermodynamics.

The term “active transport” refers to the movement of molecules across a cell membrane from a region of lower concentration to a region of higher concentration (i.e., against a concentration gradient). Unlike passive transport, which uses the kinetic energy and natural entropy of molecules moving down a gradient, active transport uses cellular energy to move them against a gradient, polar repulsion or other resistance. Active transport is usually associated with accumulating high concentrations of molecules that the cell needs, such as ions, glucose and amino acids. Specialized transmembrane proteins recognize the substance and allow it to move across the membrane when it otherwise would not, either because the phospholipid bilayer of the membrane is impermeable to the substance moved or because the substance is moved against the direction of its concentration gradient. There are two forms of active transport, primary active transport and secondary active transport. In primary active transport, the proteins involved are pumps that normally use chemical energy in the form of ATP. Secondary active transport makes use of potential energy, which is usually derived through exploitation of an electrochemical gradient.

The term “administration” refers to the introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration. In an embodiment, the bioavailability of an agent is increased by converting it to a prodrug.

The term “pharmaceutical composition” is intended to include the combination of an active agent (i.e., a prodrug therapeutic) with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

The term “pharmaceutically acceptable salt” refers to an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. In general, a pharmaceutically acceptable acid or base salt can be synthesized from a parent compound that contains a basic or acidic moiety by any conventional chemical method. For example, such salts can be prepared by reacting the free acid or base forms of these compounds with a stoichiometric amount of the appropriate base or acid in an appropriate solvent.

The term “an effective amount” refers to the amount of the defined component sufficient to achieve the desired chemical composition or the desired biological and/or therapeutic result. The desired result can be the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art.

In an embodiment, a “subject” of diagnosis or treatment is a prokaryotic or a eukaryotic cell, a tissue culture, a tissue or an animal, e.g. a mammal, including a human. Non-human animals subject to diagnosis or treatment include, for example, a simian, a murine, a canine, a leporid, such as a rabbit, livestock, sport animals, and pets.

The terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

For purposes herein, a formulation, can be administered by one or more of transdermal, intranasal, subcutaneous, intravenous, Intraperitoneal or lymphatic route, including intramuscular, subcutaneous or intradermal laminar flow injection with or without needles.

### DETAILED DESCRIPTION

Perillyl alcohol (PA) and other cyclic monoterpenes have demonstrated antineoplastic activity in cellular and animal models. The mode of action of PA in programed cell death of cancer cells is being investigated. However, studies have shown that PA can increase mannose-6-phosphate and the insulin-like growth factor II receptors. In addition, it has been demonstrated that treatment of cell lines with PA increases tissue growth factor beta receptors and BAK, a proapoptotic BCL-2 family member. PA exposure has also been shown to decrease RAS protein prenylation, decrease ubiquinone synthesis and induce Phase I and Phase II detoxification systems.

Another proposed mechanism of action of PA in cancer cell apoptosis involves regulation of CDC42 gene expression, including enzymatic interaction with rho GTPase and cessation of spindle fiber formation. Specifically, it has been proposed that the dynamic between CDC42 and response proteins γ-H2AX and p-Chk1 is altered in the presence of these compounds. There is also evidence to suggest an increase in arrestation through G2/M checkpoints may be affecting the relative levels of expression of response proteins γ-H₂AX and p-Chk1. While additional studies are needed to confirm the precise mode of action of PA in cancer cell apoptosis, the conversion of PA, limonene and related compounds to prodrugs represents a novel strategy for cancer treatment. However, the compounds have been ineffective in human models because of poor bioavailability and issues with ADME (adsorption, distribution, metabolism and excretion).

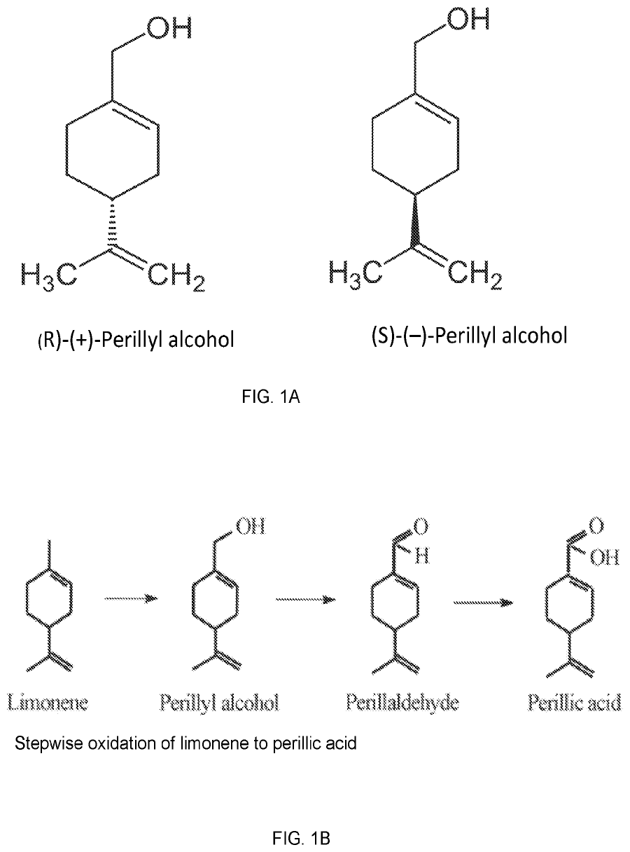

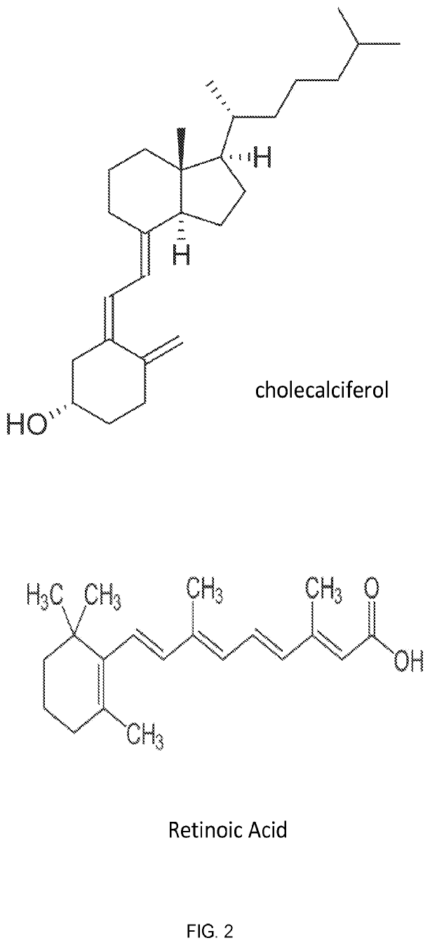

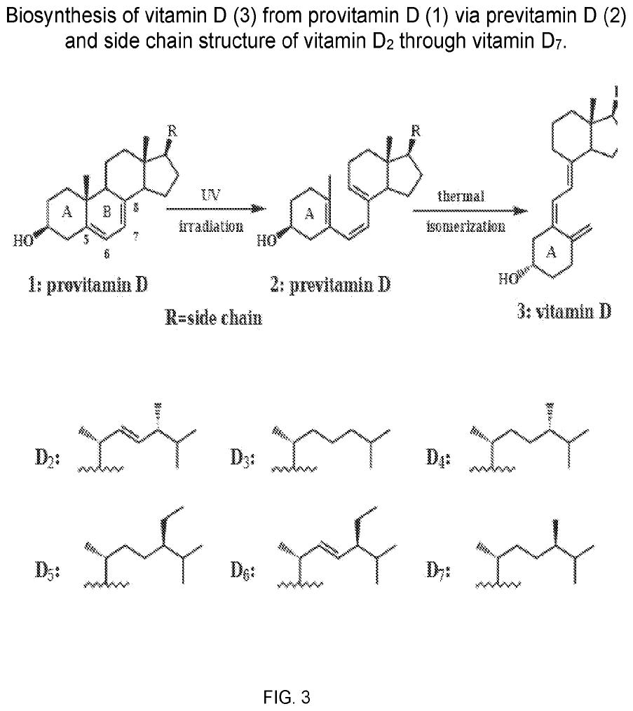

The structures of (R)-(+) PA and (S)-(−) PA are depicted in FIG. 1A. In nature, limonene is formed from geranyl pyrophosphate, via cyclization of a neryl carbocation or its equivalent. The final step involves loss of a proton from the cation to form the alkene. The stepwise oxidation of limonene to perillic acid is depicted in FIG. 1B.

A prodrug is a medication or compound that, after administration, is metabolized (i.e., converted within the body) into a pharmacologically active drug. Instead of administering a drug directly, a corresponding prodrug can be used to improve how the drug is absorbed, distributed, metabolized, and excreted (ADME). Approximately 10% of all marketed drugs worldwide can be considered prodrugs. For example, aspirin was originally synthesized in the 19th century in attempt to produce a prodrug of salicylic acid that would be tolerable when taken orally.

Since 2008, at least 30 prodrugs have been approved by the FDA. Prodrugs can be classified into two major types, based on how the body converts the prodrug into the final active drug form. Type I prodrugs are bioactivated inside the cells (i.e., intracellularly). Examples include anti-viral nucleoside analogs that must be phosphorylated and the lipid-lowering statins. Type II prodrugs are bioactivated outside cells (i.e., extracellularly) such as in digestive fluids or in the body's circulatory system. Examples of Type II prodrugs include salicin and certain antibody-, gene- or virus-directed enzyme prodrugs used in chemotherapy or immunotherapy. Both types can be further categorized into subtypes, based on factors such as (Type I) whether the intracellular bioactivation location is also the site of therapeutic action, or (Type 2) whether or not bioactivation occurs in the gastrointestinal fluids or in the circulation system.

The present invention overcomes pitfalls inherent in the prior art by providing novel compositions and methods to be used in treatment of ailments such as cancers. Specifically, the invention provides cyclic monoterpenes as prodrug compositions. As prodrugs, the bioavailability and ADME issues are improved. Also, conjugating cyclic monoterpenes can increase the lipophilicity of the compounds. This can increase penetration of the conjugate across the blood-brain barrier. Further, a lower drug dose can be administered to achieve a desired effect while minimizing toxicity.

In embodiments, the prodrugs described herein can be targeted to a specific tissue such as tumors. The compounds can be used to treat an ailment such as cancer. In other embodiments, the ailment is an infection, Parkinson's disease (PD), gallstones, cholecystitis or angiocholitis.

In embodiments, one or more cyclic monoterpenes is conjugated to a prodrug component such as cholecalciferol/Vitamin D, retinoic acid, N-methyl perillic acid, tyrosine and/or valproic acid. The molecules can be conjugated via an ester linkage. In other embodiments, a linker molecule is used to form a covalent bond. Table 1 lists several agents, their respective prodrug component and the transporter.

TABLE 1

| | Medicament/Active Agent | Prodrug Component | Transporter |
|---|---|---|---|
| 1 | Perillyl Alcohol (PA) | Retinoic Acid | Retinoic Acid |
| 2 | Perillic Acid | Cholecalciferol | Vitamin D |
| 3 | Limonene | None | None |
| 4 | Perillic Acid | Perillic Perillylate | None |
| 5 | Perillic Acid + Limonene | Retinoic Acid | Retinoic Acid |
| 6 | PA CO PA | Perillyl Carbonate | None |
| 7 | Perillic N-Methyl Amide | PANMA | FAH |
| 8 | Perillic Acid | Tyrosine | LAT1 |
| 9 | Valproic Acid/ | Valproic Acid | MCT1 |
| | Perillic Alcohol | | |

Cholecalciferol is one of the five forms of vitamin D (i.e., vitamin D3). It is naturally synthesized in skin and functions as a pro-hormone, being converted to calcitriol. Cholecalciferol is synthesized by the body and functions as a pre-hormone. Cholecalciferol is converted to its active form by two reactions: the first in the liver, the second in the kidney, to form calcitriol, whose action is mediated by the vitamin D receptor. This receptor regulates the synthesis of hundreds of enzymes and is present in virtually every cell in the body.

Retinoids are a class of compounds that comprise a four-isoprenoid unit in their molecular structures. Examples of retinoids include retinol (vitamin A), retinal, retiferol, tretinoin (all-trans-retinoic acid, e.g., retinoic acid, Retin-A), isotretinoin, alitretinoin (9-cis-retinoic acid), etretinate, acitretin, tazarotene, bexarotene and Adapalene. Retinoids and retinoid-related compounds have important and diverse functions. Retinoic acid is a metabolite of vitamin A1 (all-trans-retinol) that mediates the functions of vitamin A1 required for growth and development. All-trans-retinoic acid (ATRA) is the major occurring retinoic acid, while isomers like 13-cis- and 9-cis-retinoic acid are also present in much lower levels in mammals.

Tyrosine is a nonessential amino acid the body makes from another amino acid called phenylalanine. It is an essential component for the production of several important brain chemicals called neurotransmitters, including epinephrine, norepinephrine, and dopamine. LAT1 or SLC7A5 is a major nutrient transporter protein that is responsible for the transport of large neutral, aromatic or branched amino acids from extracellular fluids into the cells. These include many essential amino acids, such as phenyl-alanine, leucine, isoleucine, valine, tryptophan, histidine and methionine. Tyrosine is a LAT1 substrate that has a phenolic hydroxyl group amenable to conjugation with various structurally different drugs providing a biodegradable linkage.

Valproic acid is a branched short-chain fatty acid and the 2-n-propyl derivative of valeric acid. Prodrugs of valproic acid include propyl valproate (P-VPA), butyl valproate (B-VPA), isobutyl valproate (IB-VPA), isoamyl valproate (IA-VPA), and hexyl valproate (H-VPA). Because of the rapid conversion of the prodrugs to the parent drug, levels of VPA in plasma after administration of the prodrugs peak soon after administration (i.e., 6-26 minutes). Valproic acid (or sodium valproate) is commonly used to treat epilepsy and bipolar disorder and prevent migraine headache.

### Active Transport Prodrugs

There are two main modes of transport of molecules across a biological membrane: passive and active transport. Passive transport (e.g., diffusion) occurs along a concentration gradient from high to low concentration. No energy is necessary for this mode of transport. In contrast, active transport is an energy-driven process where membrane proteins transport molecules across cells. Active transport can be classified as either primary or secondary, based on how energy is coupled to fuel these mechanisms.

In embodiments, a prodrug described herein is actively transported into a target cell. A transmembrane protein can recognize the prodrug (or a portion of the prodrug) and move it across the membrane by utilizing a source of energy.

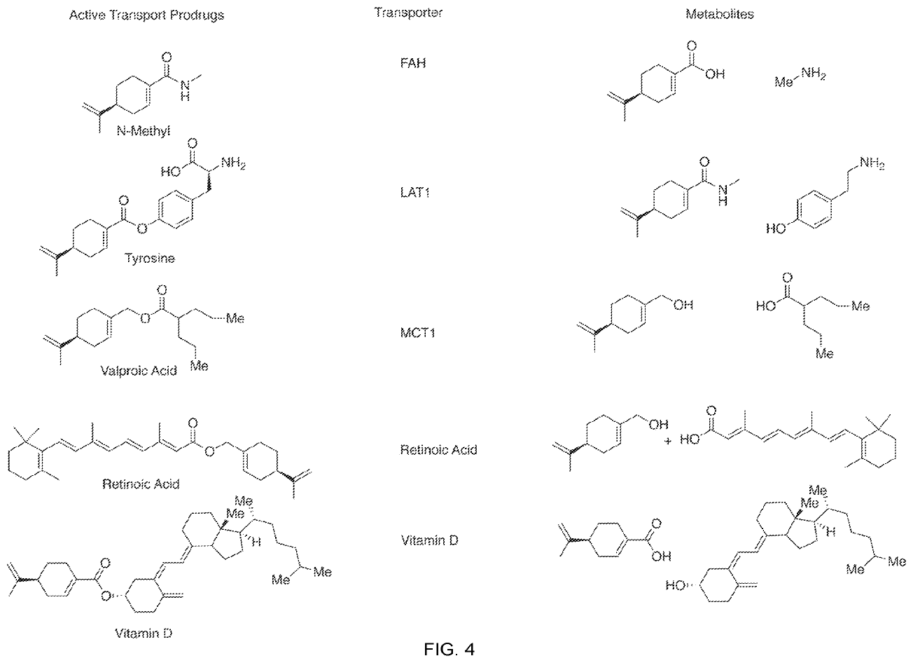

The chemical structures of several active transport prodrugs, their transporter protein and metabolites are depicted in FIG. 4. N-methyl perillic acid can be transported by FAH. The metabolites produced are perillic acid and methyl amine. Similarly, tyrosine can be conjugated to perillic acid to create a prodrug. The compound can be transported into a target cell by LAT1. Valproic acid can be conjugated to perillic acid to create a prodrug. The compound can be transported into a target cell by MCT1. Retinoic acid can be conjugated to perillic acid to create a prodrug. The compound can be transported into a target cell by a protein carrier. Further, Vitamin D can be conjugated to perillic acid to create a prodrug that can be transported into a target cell by a protein carrier.

### Passive Transport Prodrugs

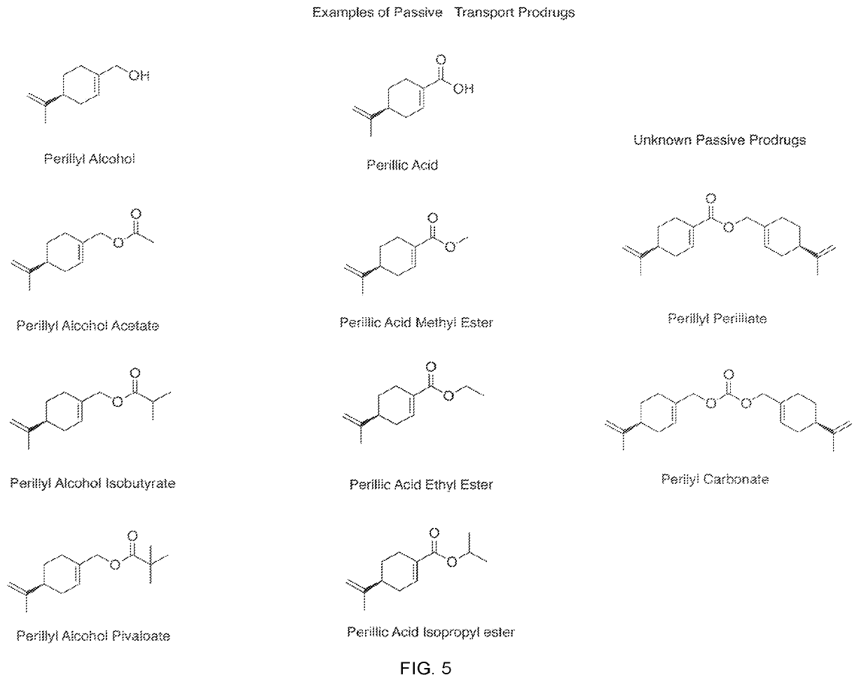

In embodiments, a prodrug described herein is passively transported into a target cell. Prodrugs that can enter a target cell via passive transport are depicted in FIG. 5 and include, for example, perillyl alcohol, perillic acid, perillyl alcohol acetate, perillic acid methyl ester, perillyl alcohol isobutyrate, perillic acid ethyl ester, perillyl alcohol pivaloate, perillic acid isopropyl ester, perillyl perilliate and perillyl carbonate.

### Methods of Treatment

Methods for treating, preventing or ameliorating a disease, disorder, a condition, or a symptom thereof or a condition related thereto are provided herein. Preferred, but non-limiting embodiments are directed to methods for treating, preventing, inhibiting or ameliorating a disease, disorder, a condition, or a symptom described below.

The present invention provides methods of treating an ailment such as cancer in a subject comprising administering to the subject one or more compounds described herein or a salt thereof, or a pharmaceutical composition of the same. In some embodiments, the subject is a subject in need of such treatment. In some embodiments, the compound is administered as a prodrug. Examples of cancer include melanoma, endometrium, lung, hematopoietic/lymphoid, ovarian, cervical, soft-tissue sarcoma, urinary tract, pancreas, thyroid, kidney, glioblastoma, breast cancer. In some embodiments, the cancer is not a B-cell proliferative type cancer. In some embodiments, the cancer is not multiple myeloma.

In any of the embodiments, one or more cancer therapies (e.g., chemotherapy, radiation therapy, immunotherapy, surgery or hormone therapy) can be co-administered further with a medicament of the invention. In one embodiment, the chemotherapeutic reagent is an alkylating agent: nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin.

Tetrazines include dacarbazine, mitozolomide and temozolomide. Aziridines include thiotepa, mytomycin and diaziquone (AZQ). Cisplatin and derivatives include cisplatin, carboplatin and oxaliplatin. In one embodiment the chemotherapeutic reagent is an anti-metabolites: the anti-folates (e.g., methotrexate), fluoropyrimidines (e.g., fluorouracil and capecitabine), deoxynucleoside analogues and thiopurines. In another embodiment the chemoptheraputic reagent is an anti-microtubule agent such as vinca alkaloids (e.g., vincristine and vinblastine) and taxanes (e.g., paclitaxel and docetaxel). In another embodiment the chemotherapeutic reagent is a topoisomerase inhibitor or a cytotoxic antibiotic such as doxorubicin, mitoxantrone, bleomycin, actinomycin, and mitomycin.

### Administration of Prodrug Medicaments

To prepare pharmaceutical compositions, one or more of monoterpene (or sesquiterpene) derivatives can be mixed with a pharmaceutical acceptable carrier, adjuvant and/or excipient, according to conventional pharmaceutical compounding techniques. Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, etc. Liquid and semisolid excipients can be, for example, glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. The compositions also can include stabilizers and preservatives.

It is understood that the present invention encompasses the use, where applicable, of stereoisomers, diastereomers and optical stereoisomers of the compounds of the invention, as well as mixtures thereof. Additionally, it is understood that stereoisomers, diastereomers, and optical stereoisomers of the compounds of the invention, and mixtures thereof, are within the scope of the invention. By way of non-limiting example, the mixture may be a racemate or the mixture may comprise unequal proportions of one particular stereoisomer over the other. Additionally, the compounds can be provided as a substantially pure stereoisomers, diastereomers and optical stereoisomers (such as epimers).

The compounds described herein can be asymmetric (e.g., having one or more stereocenters). All stereoisomers, such as enantiomers and diastereomers, are intended to be included within the scope of the invention unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds are also included within the scope of the invention and can be isolated as a mixture of isomers or as separated isomeric forms. Where a compound is capable of stereoisomerism, all such isomers are contemplated.

Compounds may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds also include hydrates and solvates, as well as anhydrous and non-solvated forms. Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

One or more of the compounds described herein can be administered by any method known in the art, including, for example, intranasal, oral, transdermal, ocular, intraperitoneal, inhalation, intravenous, ICV, intracisternal injection or infusion, subcutaneous, implant, vaginal, sublingual, urethral (e.g., urethral suppository), subcutaneous, intramuscular, intravenous, rectal, sub-lingual, mucosal, ophthalmic, spinal, intrathecal, intra-articular, intra-arterial, sub-arachinoid, bronchial and lymphatic administration. A topical formulation can be in the form of gel, ointment, cream, aerosol, etc. An intranasal formulation can be delivered as a spray or in a drop. A prodrug formulation can be administered via a transdermal patch or iontophoresis. A formulation for inhalation can be delivered using a nebulizer or similar device. Compositions can also take the form of tablets, pills, capsules, semisolids, powders, sustained release formulations, solutions, suspensions, elixirs, aerosols or any other appropriate compositions.

In another aspect, certain embodiments are directed to a sustained release drug delivery platform that releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In embodiment, the ailment is a cancer. A cancer can include one or more of lung cancer, breast cancer, colon cancer, midline carcinoma, mesenchymal tumor, liver tumor, neoplasm, neuroendocrine tumor, adrenal cancer, precancerous carcinoma, Eosinophil leukemia, acute leukemia, acute lymphoblastic leukemia, acute megakaryocytic leukemia, acute mononuclear leukemia, acute promyelocytic leukemia, adenocarcinoma, adenocarcinoma carcinoma, adenoma, squamous cell carcinoma, Adult T-cell leukemia/lymphoma, aggressive NK-cell leukemia, AIDS-related lymphoma, alveolar rhabdomyosarcoma, alveolar soft tissue sarcoma, B-cell chronic lymphocytic leukemia, B-cell lymphocytic leukemia and B-cell lymphoma. The present invention also relates to a method of treating a cancer selected from the group consisting of a cell carcinoma, a biliary cancer, a bladder cancer, a blastoma, a bone cancer, a Brenner tumor, a brown tumor, a Burkitt lymphoma, a brain cancer, a carcinoma, Neoplasms of the kidney, cell tumor of the kidneys, cytoskeletal, craniopharyngioma, skin T-cell lymphoma, cervical cancer, colorectal cancer, Degos disease, connective tissue small round cell tumor, diffuse large B-cell lymphoma, Endometrioid lymphoma, follicular lymphoma, follicular thyroid carcinoma, ganglion neurotomy, gastric cancer, stomach cancer, endometrioid carcinoma, Glioblastoma multiforme, glioma glioblastoma, Pancreatic cancer, hepatocellular carcinoma, hepatic splenic T-cell lymphoma, Hodgkin's lymphoma, non-Hodgkin's lymphoma, invasive cancer, papillary carcinoma, gallbladder cancer, gastric cancer, hair-cell leukemia, angioblastoma, head and neck cancer, Lymphoma, acute lymphoblastic leukemia, Chronic lymphocytic leukemia, liver cancer, small cell lung cancer, non-small cell lung cancer, MALT lymphoma, malignant fibrous histiocytoma, malignant peripheral neurotic tumor, malignant Triton tumor, mantle cell lymphoma, marginal B-cell lymphoma, mast cell leukemia, Tumor, Mammalian carcinoma, Metastatic carcinoma of the breast, Mixed mullerian tumor, Mucinous tumor and Mammary carcinoma. The present invention relates to the use of a compound of formula I or a pharmaceutically acceptable salt or solvate thereof for the manufacture of a medicament for the treatment or prophylaxis of a neurodegenerative disease selected from the group consisting of neuroblastoma, tumor, multiple myeloma, muscle tissue neoplasm, fungal sarcoma, mucinous liposarcoma, myxoma, mucinous sarcoma, Pituitary tumor, pituitary adenoma, pituitary adenoma, pituitary adenoma, papillary thyroid carcinoma, pineal gland neoplasm, pineal gland tumor, Lymphocytic lymphoma, primary exudative lymphoma, primary effusion lymphoma, primary peritoneal cancer, prostate cancer, pancreatic cancer, parietal carcinoma, peritoneal mesothelioma, renal cell carcinoma, retinoblastoma, rhabdomyosarcoma, squamous cell carcinoma, small cell carcinoma, stomach cancer, T-cell lymphoma, synovial sarcoma, Lymphoma, testicular cancer, ovarian cancer, thyroid cancer, transitional cell carcinoma, throat cancer, urothelial carcinoma, urogenital cancer, uveitic melanoma, uterine cancer, wart cancer, visual pathway glioma, vulvar cancer, vaginal cancer, leukemia and Wilms' tumor.

Another aspect of the present application relates to a method for treating a cell proliferative disorder. The method comprises administering to a subject in need thereof an effective amount of an agent according to the present disclosure.

Any suitable route or mode of administration can be employed for providing the patient with a therapeutically or prophylactically effective dose of the agents. Exemplary routes or modes of administration include parenteral (e.g., intravenous, intraarterial, intramuscular, subcutaneous, intratumoral), oral, topical (nasal, transdermal, intradermal or intraocular), mucosal (e.g., nasal, sublingual, buccal, rectal, vaginal), inhalation, intralymphatic, intraspinal, intracranial, intraperitoneal, intratracheal, intravesical, intrathecal, enteral, intrapulmonary, intralymphatic, intracavital, intraorbital, intracapsular and transurethral, as well as local delivery by catheter or stent.

A pharmaceutical composition comprising an agent in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

In embodiments, the compositions described herein care used in conjunction with other therapeutics as treatment for one or more of the following: fibrosarcoma, myxosarcoma, liposarcoma, chondrosarcoma, osteosarcoma, chordoma, malignant fibrous histiocytoma, hemangiosarcoma, angiosarcoma, lymphangiosarcoma mesothelioma, leukemia, aleukemic leukemia, plasmacytoma; rare malignant tumors, multiple myeloma, Hodgkin lymphoma, non-Hodgkin lymphoma, malignant tumors, leiomyosarcoma, rhabdomyosarcoma, squamous cell carcinoma, epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, grades I-III, anaplastic; glioblastoma multiforme (grade IV), neuroblastoma, medulloblastoma, malignant meningioma, malignant meningioma, malignant schwannoma, neurofibrosarcoma, parathyroid carcinoma, medullary carcinoma of thyroid, bronchial carcinoid, oat cell carcinoma, pheochromocytoma, islet cell carcinoma, malignant carcinoid, malignant paraganglioma, melanoma, malignant schwannoma, merkel cell neoplasm, cystosarcoma phylloides and/or Wilms tumor.

The therapeutic agents in the pharmaceutical compositions may be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of the recombinant vector may vary depending on the condition to be treated, the severity and course of the condition, the mode of administration, whether the antibody or agent is administered for preventive or therapeutic purposes, the bioavailability of the particular agent(s), the ability of the agent to elicit a desired response in the individual, previous therapy, the age, weight and sex of the patient, the patient's clinical history and response to the antibody, the type of the agent used, discretion of the attending physician, etc. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

As a general proposition, a therapeutically effective amount or prophylactically effective amount of the agent will be administered in a range from about 1 ng/kg body weight/day to about 100 mg/kg body weight/day whether by one or more administrations. In a particular embodiment, each agent is administered in the range of from about 1 ng/kg body weight/day to about 10 mg/kg body weight/day, about 1 ng/kg body weight/day to about 1 mg/kg body weight/day, about 1 ng/kg body weight/day to about 100 g/kg body weight/day, about 1 ng/kg body weight/day to about 10 g/kg body weight/day, about 1 ng/kg body weight/day to about 1 g/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 g/kg body weight/day, about 10 ng/kg body weight/day to about 10 mg/kg body weight/day, about 10 ng/kg body weight/day to about 1 mg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 1 mg/kg body weight/day to about 100 mg/kg body weight/day, about 1 mg/kg body weight/day to about 10 mg/kg body weight/day, about 1 mg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day, about 10 mg/kg body weight/day to about 10 mg/kg body weight/day, about 10 mg/kg body weight/day to about 1 mg/kg body weight/day, about 10 mg/kg body weight/day to about 100 mg/kg body weight/day, about 100 mg/kg body weight/day to about 100 mg/kg body weight/day, about 100 mg/kg body weight/day to about 10 mg/kg body weight/day, about 100 mg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the agent is administered at a dose of 200 g to 500 g every three days, or 25 mg/kg body weight every three days.

In other embodiments, each agent is administered in the range of about 10 ng to about 100 ng per individual administration, about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per individual administration, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 mg to about 10 mg per individual administration, about 1 mg to about 100 mg per individual administration, about 1 mg to about 5 mg per individual administration, about 1 mg to about 10 mg per individual administration, about 1 mg to about 100 mg per individual administration, about 1 mg to about 1000 mg per individual administration, about 1 mg to about 10,000 mg per individual administration, about 10 mg to about 100 mg per individual administration, about 10 mg to about 1 mg per individual administration, about 10 mg to about 50 mg per individual administration, about 10 mg to about 100 mg per individual administration, about 10 mg to about 1000 mg per individual administration, about 10 mg to about 10,000 mg per individual administration, about 100 mg to about 1 mg per individual administration, about 100 mg to about 10 mg per individual administration, about 100 mg to about 500 mg per individual administration, about 100 mg to about 1000 mg per individual administration, about 100 mg to about 10,000 mg per individual administration, about 1 mg to about 10 mg per individual administration, about 1 mg to about 100 mg per individual administration, about 1 mg to about 1000 mg per individual administration, about 1 mg to about 10,000 mg per individual administration, about 10 mg to about 100 mg per individual administration, about 10 mg to about 1000 mg per individual administration, about 10 mg to about 10,000 mg per individual administration, about 100 mg to about 1000 mg per individual administration, about 100 mg to about 5,000 mg per individual administration and about 1000 mg to about 10,000 mg per individual administration. The agent may be administered daily, every 2, 3, 4, 5, 6 or 7 days, or every 1, 2, 3 or 4 weeks.

In other embodiments, each agent is administered in the range of about 10 ng to about 100 ng per injection, about 10 ng to about 1 g per injection, about 10 ng to about 10 g per injection, about 10 ng to about 100 mg per injection, about 10 ng to about 1 mg per injection, about 10 ng to about 10 mg per injection, about 10 ng to about 100 mg per injection, about 10 ng to about 1000 mg per injection, about 10 ng to about 10,000 mg per injection, about 100 ng to about 1 mg per injection, about 100 ng to about 10 mg per injection, about 100 ng to about 100 mg per injection, about 100 ng to about 1 mg per injection, about 100 ng to about 10 mg per injection, about 100 ng to about 100 mg per injection, about 100 ng to about 1000 mg per injection, about 100 ng to about 10,000 mg per injection, about 1 mg to about 10 mg per injection, about 1 mg to about 100 mg per injection, about 1 mg to about 5 mg per injection, about 1 mg to about 10 mg per injection, about 1 mg to about 100 mg per injection, about 1 mg to about 1000 mg per injection, about 1 mg to about 10,000 mg per injection, about 10 mg to about 100 mg per injection, about 10 mg to about 1 mg per injection, about 10 mg to about 50 mg per injection, about 10 mg to about 100 mg per injection, about 10 mg to about 1000 mg per injection, about 10 mg to about 10,000 mg per injection, about 100 mg to about 1 mg per injection, about 100 mg to about 10 mg per injection, about 100 mg to about 500 mg per injection, about 100 mg to about 1000 mg per injection, about 100 mg to about 10,000 mg per injection, about 1 mg to about 10 mg per injection, about 1 mg to about 100 mg per injection, about 1 mg to about 1000 mg per injection, about 1 mg to about 10,000 mg per injection, about 10 mg to about 100 mg per injection, about 10 mg to about 1000 mg per injection, about 10 mg to about 10,000 mg per injection, about 100 mg to about 1000 mg per injection, about 100 mg to about 5,000 mg per injection and about 1000 mg to about 10,000 mg per injection. The agent may be injected hourly, bi-hourly, tri-hourly, every 90 minutes, every two hours, every three hours, every four hours, twice daily, three times daily, four times daily, five times daily, six times daily, seven times daily, eight times daily, every 2, 3, 4, 5, 6 or 7 days, or every 1, 2, 3 or 4 weeks.

In other particular embodiments, the amount of the agent may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient.

In other aspects of this embodiment, a pharmaceutical composition disclosed herein reduces the size of a tumor by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein is in an amount sufficient to allow customary administration to an individual. In aspects of this embodiment, a pharmaceutical composition disclosed herein may be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition. In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a pharmaceutical composition. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A pharmaceutical composition disclosed herein may comprise a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition disclosed herein may be of any concentration desired. In an aspect of this embodiment, the final concentration of a pharmaceutical composition may be a therapeutically effective amount. In other aspects of this embodiment, the final concentration of a pharmaceutical composition in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of a pharmaceutical composition disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the final concentration of a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from cancer. As used herein, the term “treating,” refers to reducing or eliminating in an individual a clinical symptom of cancer; or delaying or preventing in an individual the onset of a clinical symptom of cancer. For example, the term “treating” can mean reducing a symptom of a condition characterized by a cancer, including, but not limited to, tumor size, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with cancer are well known and can be determined by a person of ordinary skill in the art by taking into account factors, including, without limitation, the location of the cancer, the cause of the cancer, the severity of the cancer, and/or the tissue or organ affected by the cancer. Those of skill in the art will know the appropriate symptoms or indicators associated with a specific type of cancer and will know how to determine if an individual is a candidate for treatment as disclosed herein.

In another aspect, a pharmaceutical composition disclosed herein reduces the severity of a symptom of a disorder associated with a cancer. In aspects of this embodiment, a pharmaceutical composition disclosed herein reduces the severity of a symptom of a disorder associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces the severity of a symptom of a disorder associated with a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In aspects of this embodiment, a therapeutically effective amount of a pharmaceutical composition disclosed herein reduces a symptom associated with cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces a symptom associated with cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces a symptom associated with cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a pharmaceutical composition disclosed herein generally is in the range of about 0.001 mg/kg/day to about 100 mg/kg/day. In aspects of this embodiment, an effective amount of a pharmaceutical composition disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day. In other aspects of this embodiment, an effective amount of a pharmaceutical composition disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day. In yet other aspects of this embodiment, an effective amount of a pharmaceutical composition disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day. In still other aspects of this embodiment, an effective amount of a pharmaceutical composition disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

Dosing can be single dosage or cumulative (serial dosing), and can be readily determined by one skilled in the art. For instance, treatment of a cancer may comprise a one-time administration of an effective dose of a pharmaceutical composition disclosed herein. Alternatively, treatment of a cancer may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a cancer therapeutic disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a cancer therapeutic is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a cancer therapeutic and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a cancer therapeutic is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a cancer therapeutic disclosed herein reduces or maintains a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a cancer therapeutic disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a cancer therapeutic disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A pharmaceutical composition or cancer therapeutic is administered to an individual. An individual is typically a human being, but can be an animal, including, but not limited to, dogs, cats, birds, cattle, horses, sheep, goats, reptiles and other animals, whether domesticated or not. Typically, any individual who is a candidate for treatment is a candidate with some form of cancer, whether the cancer is benign or malignant, a tumor, solid or otherwise, a cancer cell not located in a tumor or some other form of cancer. Among the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

In one aspect, a pharmaceutical composition disclosed herein reduces a symptom of a disorder associated with a cancer (e.g., tumor size and/or number of tumor cells). In aspects of this embodiment, a pharmaceutical composition disclosed herein reduces a symptom of a disorder associated with a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces a symptom of a disorder associated with a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In one aspect, a pharmaceutical composition disclosed herein reduces a symptom of a disorder associated with an ailment (e.g., an infection, Parkinson's disease (PD), gallstones, cholecystitis or angiocholitis). In aspects, a pharmaceutical composition disclosed herein reduces a symptom of a disorder associated with the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces a symptom of a disorder associated with an ailment by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In another aspect, a pharmaceutical composition disclosed herein reduces the frequency of a symptom of a disorder associated with a cancer incurred over a given time period. In aspects of this embodiment, a pharmaceutical composition disclosed herein reduces the frequency of a symptom of a disorder associated with a cancer (e.g., such as tumor size) incurred over a given time period by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces the frequency of a symptom of a disorder associated with a cancer incurred over a given time period by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In embodiments, the prodrugs can cross the blood-brain barrier. In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of the prodrug crosses the blood brain barrier.

In embodiments, the prodrugs increase the half-life of a small molecule active agent that is administered to a subject. In some embodiments, the half-life is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% compared to a non-conjugated active agent.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

Dosing can be single dosage or cumulative (serial dosing), and can be readily determined by one skilled in the art. A prodrug formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a prodrug formulation as disclosed herein.

Alternatively, treatment of a disease may comprise multiple administrations of an effective dose of a prodrug formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a prodrug formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a prodrug formulation disclosed herein that is administered can be adjusted accordingly. In one embodiment, a prodrug formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

The appropriate effective amount of a prodrug formulation disclosed herein to be administered to an individual can be determined by a person of ordinary skill in the art by taking into account factors, including, without limitation, an improvement in the individual based upon one or more clinical symptoms, and/or physiological indicators associated with improvements in muscle performance, reduced soreness and/or overall health, the particular characteristics, history and risk factors of the patient, such as, e.g., age, weight, general health and the like, or any combination thereof. Additionally, where repeated administration of a prodrug delivery formulation is used, an effective amount of a prodrug delivery formulation will further depend upon factors, including, without limitation, the frequency of administration, the half-life of the prodrug delivery formulation, or any combination thereof. It is known by a person of ordinary skill in the art that an effective amount of a prodrug delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For example, oral administration of a prodrug formulation disclosed herein generally would be expected to require higher dosage levels than administration by inhalation. Similarly, systemic administration of a prodrug formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, a reduction or alleviation of symptoms of an ailment such as muscle cramping or soreness in an individual. As used herein, the term “treating,” refers to reduction in size, number and/or area of tumors. For example, the term “treating” can mean reduction of tumor size by, e.g., at least 5%, at least 10%, at least 15%, least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with an ailment such as cancer and will know how to determine if an individual is a candidate for treatment as disclosed herein.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Conjugation of Perillyl Alcohol to Cholecalciferol

In this example, perillyl alcohol (PA) is conjugated to cholecalciferol. The molecules can be conjugated via an ester linkage using methods known in the art.

### Example 2

### Conjugated Perillyl Alcohol for Treatment of Glioblastoma

In this example, a patient seeks treatment for glioblastoma. A health care professional suggests that the patient try perillyl alcohol (PA). The PA is administered orally to the patient as a prodrug. Specifically, PA is conjugated to cholecalciferol.

The prodrug formulation presents several advantages including increased stability, increased solubility and increased lipophilicity. The formulation also allows for selective targeting to tumor cells and permeability across the blood-brain barrier.

The health care professional monitors the size of the tumor during the course of treatment. In this example, a therapeutic amount of the prodrug formulation is administered daily over the course of six weeks.

### Example 3

### Conjugation of Perillyl Alcohol to Retinoic Acid

In this example, perillyl alcohol (PA) is conjugated to retinoic acid. The molecules can be conjugated via an ester linkage using methods known in the art.

### Example 4

### Conjugated Perillyl Alcohol for Treatment of Glioblastoma

In this example, a patient seeks treatment for glioblastoma. A health care professional suggests that the patient try perillyl alcohol (PA). The PA is administered orally to the patient as a prodrug. Specifically, PA is conjugated to retinoic acid.

The prodrug formulation presents several advantages including increased stability, increased solubility and increased lipophilicity. The formulation also allows for selective targeting to tumor cells and permeability across the blood-brain barrier.

The health care professional monitors the size of the tumor during the course of treatment. In this example, a therapeutic amount of the prodrug formulation is administered daily over the course of six weeks.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the disease, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the prodrug disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and preferably is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the peptide is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

In still another aspect, the present specification provides a method for preventing or treating of cancer, infectious diseases or neurodegenerative diseases comprising the step of administering to a subject the chimeric protein or the pharmaceutical composition including the same.

Given the teachings and guidance provided herein, those skilled in the art will understand that a formulation described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

In various embodiments, a formulation can include, without limitation, combinations of bioactive agents (such as viruses, proteins, antibodies, peptides and the like as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

In various embodiments, a formulation can include one or more preservatives and/or additives known in the art. Similarly, a formulation can further be formulated, without limitation, into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition. The composition can be a solid, a semisolid or an aerosol and a pharmaceutical compositions is formulated as a tablet, geltab, lozenge, orally dissolved strip, capsule, syrup, oral suspension, emulsion, granule, sprinkle or pellet.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tabletting machine, the active ingredients in a free-flowing form such as a powder or granules, optionally mixed with a binder (for example, without limitation, povidone, gelatin, hydroxypropylmethyl cellulose), lubricant, inert diluent, preservative, disintegrant (for example, without limitation, sodium starch glycolate, cross-linked povidone, cross-linked sodium carboxymethyl cellulose) and/or surface-active or dispersing agent.

In an embodiment, molded tablets are made, for example, without limitation, by molding in a suitable tabletting machine, a mixture of powdered compounds moistened with an inert liquid diluent. In an embodiment, the tablets may optionally be coated or scored, and may be formulated so as to provide slow or controlled release of the active ingredients, using, for example, without limitation, hydroxypropylmethyl cellulose in varying proportions to provide the desired release profile. In an embodiment, tablets may optionally be provided with a coating, without limitation, such as a thin film, sugar coating, or an enteric coating to provide release in parts of the gut other than the stomach. In an embodiment, processes, equipment, and toll manufacturers for tablet and capsule making are well-known in the art.

In an embodiment, capsule formulations can utilize either hard or soft capsules, including, without limitation, gelatin capsules or vegetarian capsules such as those made out of hydroxymethylpropylcellulose (HMPC). In an embodiment, a type of capsule is a gelatin capsule. In an embodiment, capsules may be filled using a capsule filling machine such as, without limitation, those available from commercial suppliers such as Miranda International or employing capsule manufacturing techniques well-known in the industry, as described in detail in Pharmaceutical Capules, 2.sup.nd Ed., F. Podczeck and B. Jones, 2004. In an embodiment, capsule formulations may be prepared, without limitation, using a toll manufacturing center such as the Chao Center for Industrial Pharmacy & Contract Manufacturing, located at Purdue Research Park.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, and particularly pharmaceutical virus formulations.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 3 to about 9, about 4 to about 9, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1.-12. (canceled)

13. A prodrug comprised of limonene, perillyl alcohol, perillic acid and cholecalciferol, a pharmaceutically acceptable salt, a solvate, a tautomer, an isomer or a deuterated analog thereof.

14. A method of treating an ailment comprised of administering a therapeutic amount of the prodrug of claim 13 to a subject.

15. The method of claim 14, wherein the ailment is cancer or Parkinson's disease.

16. The method of claim 15, wherein the cancer is astrocytoma, lung cancer, non-small cell lung cancer, mesothelioma, brain cancer, glioblastoma, skin cancer, or melanoma.

17.-20. (canceled)

21. A prodrug comprised of perillyl alcohol, cholecalciferol and retinoic acid, a pharmaceutically acceptable salt, a solvate, a tautomer, an isomer or a deuterated analog thereof.

22. A method of treating an ailment comprised of administering a therapeutic amount of the prodrug of claim 21 to a subject.

23. The method of claim 22, wherein the ailment is cancer or Parkinson's disease.

24. The method of claim 23, wherein the cancer is astrocytoma, lung cancer, non-small cell lung cancer, mesothelioma, brain cancer, glioblastoma, skin cancer, or melanoma.

25.-56. (canceled)

57. A method of treating cancer in a subject, the method comprising administering a compound comprised of an active agent and a prodrug component,
wherein the active agent is a cyclic monoterpene.

58. The method of claim 57, wherein the cyclic monoterpene is perillyl alcohol, limonene, perillic acid, perillyl alcohol acetate, perillic acid methyl ester, perillyl alcohol isobutyrate, perillic acid ethyl ester, perillyl alcohol pivaloate, perillic acid isopropyl ester, perillyl perilliate or perillyl carbonate.

59. The method of claim 57, wherein the prodrug component is cholecalciferol/Vitamin D, retinoic acid, N-methyl perrilic acid, tyrosine or valproic acid.

60. The method of claim 57, wherein the cancer is astrocytoma, lung cancer, non-small cell lung cancer, mesothelioma, brain cancer, glioblastoma, skin cancer, or melanoma.

61. The method of claim 57, further comprising administration of a second cancer therapy, the second cancer therapy selected from chemotherapy, radiation therapy, immunotherapy, surgery and hormone therapy.

62.-67. (canceled)
